(19) United States
(12) Patent Application Publication
(10) Pub. No.: US 2020/0273580 A1
(43) Pub. Date: Aug. 27, 2020

(54) AI POWERED, FULLY INTEGRATED, END-TO-END RISK ASSESSMENT PROCESS TOOL

(71) Applicants: Theodore John Kaszuba, Danbury, CT (US); Jing Qu, Ontario (CA)

(72) Inventors: Theodore John Kaszuba, Danbury, CT (US); Jing Qu, Ontario (CA)

(21) Appl. No.: 16/795,725

(22) Filed: Feb. 20, 2020

Related U.S. Application Data
(60) Provisional application No. 62/808,418, filed on Feb. 21, 2019.

(51) Int. Cl. G16H 50/30; G06T 7/20; G06Q 10/06; G06K 9/62; G06N 3/08; G06N 20/00

(52) U.S. Cl. CPC G16H 50/30; G06T 7/20; G06Q 10/063118; G06T 2207/10016; G06N 3/08; G06N 20/00; G06T 2207/30196; G06K 9/6289

(57) ABSTRACT

A process comprising recording a video of an employee performing a job; uploading the video to a system having at least one of a local database and a cloud database; analyzing the video and capturing and identifying motion; and creating a physical demand analysis based on the motion.

### CROSS-REFERENCE TO RELATED APPLICATION

This application claims priority to U.S. Application No. 62/808,418 filed Feb. 21, 2019.

### BACKGROUND

The present disclosure is directed to a process that utilizes artificial intelligence biomechanical analysis that considers all motions, forces, and repetitions of a workers task to be captured and analyzed to identify the overall risk of a task by use of a scoring system and the specific motions and tasks of concern.

Every day the workforce is at risk of injury. Because traditional risk assessment methods are costly, complicated, time consuming and intrusive to the daily activities of the workforce. Risk assessments at the shop floor level are not performed on a comprehensive level. Unfortunately, and specifically for the workforce, the risk assessments are invariably prioritized by the “low hanging fruit” method.

The risk assessments that require the least costly ergonomic solutions are prioritized to the top of the list, while other risk assessments that require more costly solutions typically languish at the bottom of the list waiting to be funded, if ever. Traditional, complicated and unsustainable top-down ergonomic safety program management approach do not provide the level of detail needed.

What is needed is an accurate risk assessment tool that combines with the most efficient, effective and sustainable ergonomic program management.

### SUMMARY

In accordance with the present disclosure, there is provided a process comprising recording a video of an employee performing a job; uploading the video to a system having at least one of a local database and a cloud database; analyzing the video and capturing and identifying motion; and creating a physical demand analysis based on the motion and creating a physical demand analysis based on the motion, using the posture and material handling data collected to create advanced ergonomic risk evaluation models by body parts and also use these risk data and injury data from operator's work place to create robust predictive ergonomic risk model.

A further embodiment of any of the foregoing embodiments may additionally and/or alternatively include the motion is selected from the group consisting of kneeling, crawling, climbing, squatting, lifting, carrying, pushing and pulling. Gripping, pinch etc., also body postures such as shoulder flexion, extension, abduction, twisting, adduction, back flexion, extension, side bend and back twisting, neck flexion, extension, neck side bend and neck twisting, elbow flexion, supination, pronation etc. walking, standing, sitting, bending, reaching, lifting, carrying, pushing and pulling.

A further embodiment of any of the foregoing embodiments may additionally and/or alternatively include the process further comprises using the physical demand analysis for return to work authorizations and job placement accommodations.

A further embodiment of any of the foregoing embodiments may additionally and/or alternatively include the process further comprises generating a series of ergonomic risk assessment reports.

A further embodiment of any of the foregoing embodiments may additionally and/or alternatively include the process further comprises analyzing specific dynamic human joint motions; and recording postures, angles, distances, frequencies, and durations by individual body parts to produce a comprehensive risk assessment. Including the production of graphics of bio-mechanical analysis of continuous force throughout the duty cycle.

A further embodiment of any of the foregoing embodiments may additionally and/or alternatively include the process further comprises analyzing the motion as well as forces, and repetitions; and identifying an overall risk of a job for the specific motion and tasks of concern.

A further embodiment of any of the foregoing embodiments may additionally and/or alternatively include the system is configured to mitigate risks at an early stage by suggesting job rotation, and equipment solutions to eliminate the risks before an injury occurs.

A further embodiment of any of the foregoing embodiments may additionally and/or alternatively include multiple tasks in a day, cumulative fatigue and individual operator characteristics and biometrics can are used to perform more complex predictive modeling using the powerful data produced by the artificial intelligence.

A further embodiment of any of the foregoing embodiments may additionally and/or alternatively include the data produced by the system are used to create reports to predict risks and future WC losses based on actual client job risks, employee demographics (age, tenure, past injuries, etc.) and past losses.

A further embodiment of any of the foregoing embodiments may additionally and/or alternatively include the process further comprises integrating ergonomic science into the system to enable the artificial intelligence to specifically identify the jobs of concerns and the root causes to mitigate the risks.

A further embodiment of any of the foregoing embodiments may additionally and/or alternatively include the process further comprises outputting reports that identify risk by body part (hands, wrists, elbows, shoulder, back, neck and legs) and color coding the risk green, yellow or red based on the risk factors identified.

A further embodiment of any of the foregoing embodiments may additionally and/or alternatively include the process further comprises calculating an exposure score based on the number of hours per day or week the job is performed.

A further embodiment of any of the foregoing embodiments may additionally and/or alternatively include the process further comprises creating Management reports and dashboards configured to allow for tracking of jobs, root causes, and solution implementation across a site or organization.

A further embodiment of any of the foregoing embodiments may additionally and/or alternatively include the process further comprises identifying jobs, employees and clients of concern; and developing strategies to control the losses and improve underwriting endeavors.

A further embodiment of any of the foregoing embodiments may additionally and/or alternatively include the process further comprises creating and collecting detailed task related biomechanical information; and directly saving the detailed task related biomechanical information to a database.

A further embodiment of any of the foregoing embodiments may additionally and/or alternatively include the process further comprises combining the task related biomechanical information with the past injury and loss history using a deep neural network and machine learning; and analyzing and recognizing complex human behavior patterns and expected injuries and losses.

A further embodiment of any of the foregoing embodiments may additionally and/or alternatively include the process further comprises suggesting job rotation, equipment solutions and specific ways to eliminate the risks before the injury occurs.

Other details of the process are set forth in the following detailed description and the accompanying drawings wherein like reference numerals depict like elements.

### BRIEF DESCRIPTION OF THE DRAWINGS

FIG. 1 is a schematic diagram of part of an exemplary process disclosed herein.

FIG. 2 is a schematic diagram of part of the exemplary process disclosed herein.

FIG. 3 is a schematic diagram of a stick figure used to find angles of posture for the exemplary process disclosed herein.

FIG. 4 is a schematic diagram of part of the exemplary process disclosed herein.

FIG. 5 is a schematic diagram of part of the exemplary process disclosed herein.

FIG. 6 is a schematic diagram of an exemplary system architecture.

### DETAILED DESCRIPTION

The process is a productive and sustainable tool for use at the shop floor level, and consists of four important attributes.

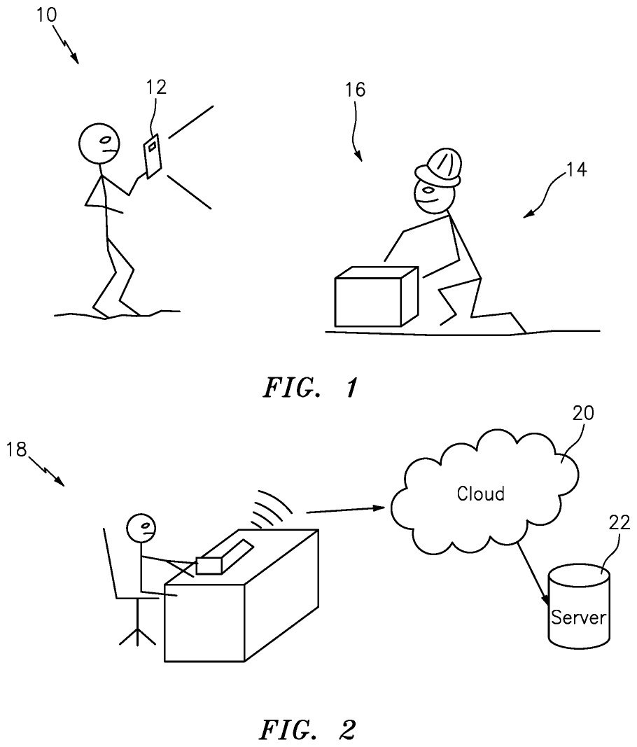

As seen at FIG. 1, anyone with an aptitude to use a camera and a computer can easily use the risk assessment process 10. Using a simple phone camera 12, a video of an employee 14 performing a job is recorded 16. There are specific instructions and techniques on how to effectively take a video for ergonomic risk assessment.

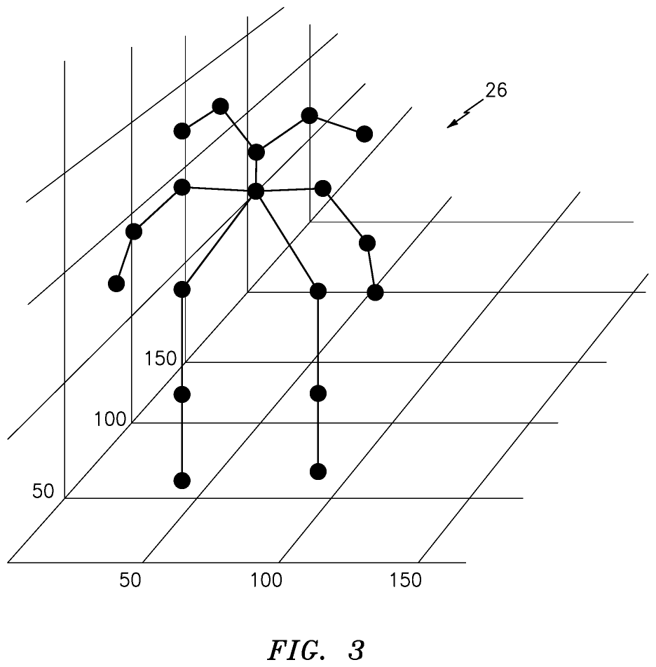

As seen in FIG. 2, once the video is complete, the video is uploaded 18 to an AI engine on a cloud 20 or a local server database 22. When uploading the data, the job/task information is entered. The information can include job name, location, operator identification and height and weight. At FIG. 3, the system's powerful artificial intelligence (AI) technology 24 analyzes the video and captures and identifies specific motions, such as walking, standing, sitting, bending, reaching, lifting, carrying, pushing and pulling, etc. A 3D computer vision deep learning model, can be used to produce a stick man 26 in 3D. The 3D deep learning model can be trained by using large volume of videos/images of human poses along with its 3D coordinates, to predict the 3D posture data and stick man 26 using 2D video/images captured through normal camera, such as cell phone camera 12. Once the stick man 26 has been developed, mathematical formulas can be applied to calculate body part angles for postures. Then risk assessment reports are created based on the angles and actions of the operator.

These motions are used to create the physical demand analysis (PDA), or physical demands analysis report 28 of job demands that can be used for return to work authorizations and job placement accommodations 30. In addition to the PDA reports, the system also generates a series of ergonomic risk assessment reports 32.

Using the motions that were captured, the system further analyzes the specific dynamic human joint motions and records postures, angles, distances, frequencies, and durations by individual body parts to produce the most advanced and comprehensive risk assessment in minutes. In an exemplary embodiment, mathematical formulas and computer vision deep learning models are used to recognize mobility such as kneeling, bending, walking, standing, sitting, bending, reaching, crawling, climbing, squatting, lifting, carrying, pushing and pulling. Gripping, pinch, also body postures such as Shoulder flexion, extension, abduction, twisting, adduction, Back flexion, extension, side bend and back twisting, neck flexion, extension, neck side bend and neck twisting, Elbow flexion, supination, pronation and the like.

Multiple tasks in a day, cumulative fatigue and individual operator characteristics and biometrics can also be used to perform more complex predictive modeling using the powerful data produced by artificial intelligence to finally get an answer to the most complex questions 34. This level of analysis is traditionally only performed by highly skilled professional ergonomists using very expensive instrumentation and sensors.

The ergonomic science is integrated into the software to enable the AI to specifically identify the jobs of concerns and the root causes to mitigate the risks. This invariably saves companies valuable time and money, and those recouped resources can be reallocated to the ergonomic solution phase.

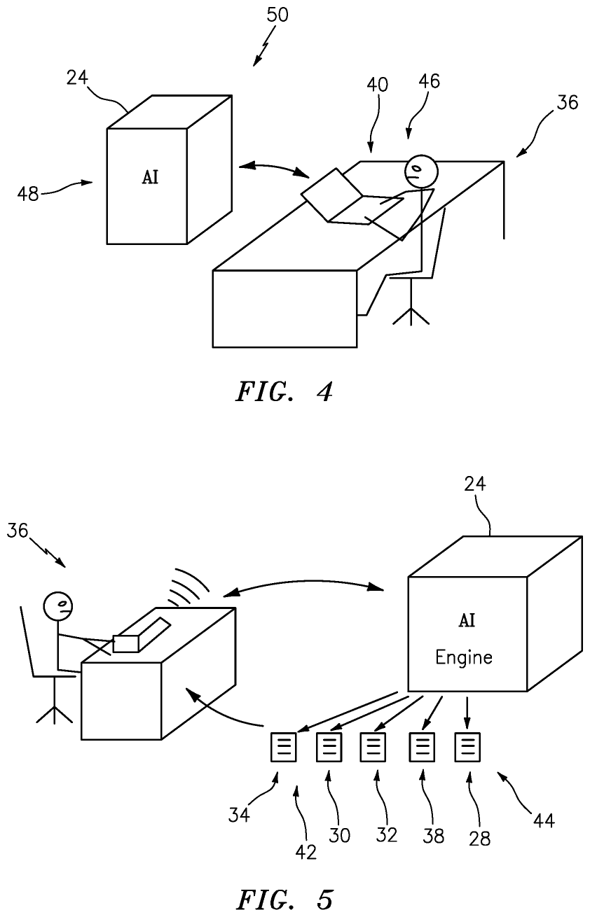

FIG. 4, an analyst 36 can verify the results provided by the system's AI technology.

At FIG. 5 the AI engine 24 is shown creating the reports for an analyst to analyze.

The power of AI easily and quickly creates an accurate and affordable risk assessment repots for all. Gone are the days of spreadsheets, forms, counting motions, performing complex calculations, only analyzing the most difficult motion, and memorizing confusing medical and biomechanical terms.

The disclosed process allows for exponentially more risk assessments to be completed and freeing up the time for the analyst 36 or risk control consultant to do other things.

The output of the system 24 are reports 38 that identify risk by body part (hands, wrists, elbows, shoulder, back, neck and legs) and color codes the risk green, yellow or red based on the risk factors identified. In addition, an exposure score 40 is calculated and based on the number of hours per day or week the job is performed. This allows for risk profiles of a series of jobs an employee may perform over the course of the day or week. Management reports and dashboards allow for tracking of jobs, root causes, and solution implementation across a site or organization 42.

In addition to the standard risk assessment and management reports, the data produced by the disclosed system can be used for other reports to predict risks and future WC losses based on actual client job risks, employee demographics (age, height, weight, gender, tenure, past injuries, etc.) and past losses.

Combining this additional big data set into the system software would allow companies to specifically identify jobs, employees and clients of concern and develop strategies to effectively control their losses and improve underwriting endeavors 44.

Through the application of artificial intelligence (AI) and machine learning, integrated with the science of ergonomics, a fast, accurate, and easy risk assessment methodology is disclosed that is repeatable, sustainable and affordable for all.

The disclosed system software creates and collects detailed task related biomechanical information and directly saves this detailed data to data bases 46. Imagine now that you have hundreds of millions of human postures, and biomechanical and demographic data at your fingertips. The data with the past injury and loss history can be combined using most advance deep neural network and machine learning to analyze and recognize these complex human behavior patterns and expected injuries and losses 48.

This data will greatly improve future predictive models for loss ratio and reserve calculation. Most importantly is that with more accurate predictions of future injuries, the system can help companies to mitigate the risks at an early stage by suggesting job rotation, equipment solutions and other ways to eliminate the risks before the injury occurs 50. In the long run this will drive down losses, and insurers will have potential to offer lower insurance premiums to customers that use the system.

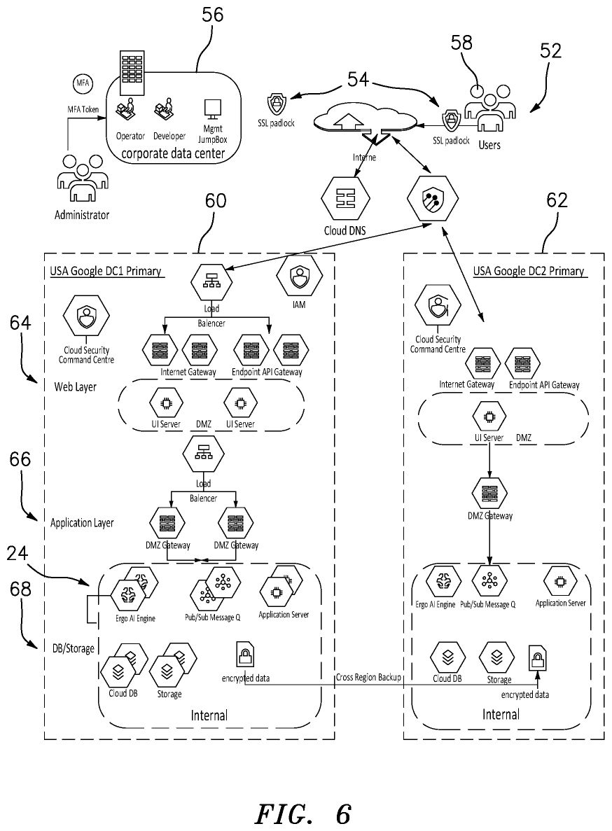

FIG. 6 is a schematic diagram of an exemplary system architecture 52. The system architecture 52 includes various communications links 54 between corporate data center 56 the internet and users. The system architecture 52 includes a primary 60 and backup 62 cloud servers. There are various layers within the server 60 such as web layer 64, application layer 66 and storage layer 68. The ergo AI engine 24 is in the application layer 66.

Dynamic AI biomechanical analysis allows for all motions, forces, and repetitions to be captured and analyzed to identify the overall risk of a job using our proprietary scoring system and the specific motions and tasks of concern.

Companies will benefit from the risk assessments and PDA's generated by the disclosed process. The disclosed process will provide companies with the tools that will allow them achieve a decisive advantage over their competitors, through the implementation of results oriented, measurable and sustainable ergonomic safety programs in their facilities.

The disclosed process will improve worker lives by creating a safer work environment that dramatically reduces or eliminates task related injuries. At the same time process will improve company's bottom line by proactively mitigating, or altogether eliminating WC claims.

The disclosed process can produce risk assessment and physical demand analysis (PDA's) that will produce very accurate, and scientifically based big data that can be used in many ways to create more accurate predictive models for expected loss ratios, more accurate estimation of cost trend factors, more accurate loss development factors, and more accurate projections of future net losses, resulting in possible justification for cash reserves.

The disclosed process will provide the data format that will provide ergonomic factory equipment solutions to reduce or eliminate the risk of injury to operators, due to repetitive or excessive, lifting bending, pushing and pulling while performing tasks that they are assigned throughout the course of their day.

There has been provided a process. While the process has been described in the context of specific embodiments thereof, other unforeseen alternatives, modifications, and variations may become apparent to those skilled in the art having read the foregoing description. Accordingly, it is intended to embrace those alternatives, modifications, and variations which fall within the broad scope of the appended claims.

What is claimed is:

1. A risk assessment process comprising:
recording a video of an employee performing a job;
uploading the video to a system having at least one of a local database and a cloud database;
analyzing the video and capturing and identifying motion; and
creating a physical demand analysis based on the motion.

2. The process according to claim 1, wherein said motion is selected from the group consisting of walking, standing, sitting, bending, reaching, lifting, carrying, pushing and pulling, kneeling, crawling, climbing, squatting, lifting, gripping, pinch, body postures such as shoulder flexion, extension, abduction, twisting, adduction, back flexion, extension, side bend and back twisting, neck flexion, extension, neck side bend and neck twisting, elbow flexion, supination, pronation and the like.

3. The process according to claim 1, further comprising:
using the physical demand analysis for return to work authorizations and job placement accommodations.

4. The process according to claim 1, further comprising:
generating a series of ergonomic risk assessment reports.

5. The process according to claim 1, further comprising:
analyzing specific dynamic human joint motions; and
recording postures, angles, distances, frequencies, and durations by individual body parts to produce a comprehensive risk assessment.

6. The process according to claim 1, further comprising:
analyzing said motion as well as forces, and repetitions; and
identifying an overall risk of a job for the specific motion and tasks of concern.

7. The process according to claim 1, wherein said system is configured to mitigate risks at an early stage by suggesting job rotation, and equipment solutions to eliminate the risks before an injury occurs.

8. The process according to claim 1, wherein multiple tasks in a day, cumulative fatigue and individual operator characteristics and biometrics can are used to perform more complex predictive modeling using the powerful data produced by the artificial intelligence.

9. The process according to claim 1, wherein the data produced by the system are used to create reports to predict risks and future WC losses based on actual client job risks, employee demographics (age, tenure, weight, height, past injuries, etc.) and past losses.

10. The process according to claim 1 further comprising:
integrating ergonomic science into the system to enable the artificial intelligence to specifically identify the jobs of concerns and the root causes to mitigate the risks.

11. The process according to claim 1, further comprising:
outputting reports that identify risk by body part (hands, wrists, elbows, shoulder, back, neck and legs) and color coding the risk green, yellow or red based on the risk factors identified.

12. The process according to claim 1, further comprising:
calculating an exposure score based on the number of hours per day or week the job is performed.

13. The process according to claim 1, further comprising:
creating Management reports and dashboards configured to allow for tracking of jobs, root causes, and solution implementation across a site or organization.

14. The process according to claim 1, further comprising:
identifying jobs, employees and clients of concern; and
developing strategies to control the losses and improve underwriting endeavors.

15. The process according to claim 1, further comprising:
creating and collecting detailed task related biomechanical information; and
directly saving said detailed task related biomechanical information to a database.

16. The process according to claim 15, further comprising:
combining the task related biomechanical information with the past injury and loss history using a deep neural network and machine learning; and
analyzing and recognizing complex human behavior patterns and expected injuries and losses.

17. The process according to claim 16, further comprising:
suggesting job rotation, equipment solutions and specific ways to eliminate the risks before the injury occurs.
